(19) United States
(12) Patent Application Publication
(10) Pub. No.: US 2022/0397573 A1
(43) Pub. Date: Dec. 15, 2022

(54) Biomolecular Detection Device

(71) Applicants: Hoffmann-La Roche Inc., Little Falls, NJ (US); ETH Zürich, Zurich (CH)

(72) Inventors: Yves Lukas Blickenstorfer, Zurich (CH); Christof Fattinger, Basel (CH); Andreas Frutiger, Zurich (CH); Andreas Michael Reichmuth, Zurich (CH); Janos Voeroes, Zurich (CH)

(21) Appl. No.: 17/642,776

(22) PCT Filed: Sep. 16, 2020
(86) PCT No.: PCT/EP2020/075914

§ 371 Date: Mar. 14, 2022

(30) Foreign Application Priority Data
Sep. 17, 2019 (EP) 19197858.4

(51) Int. Cl. G01N 33/543; G01N 15/14

(52) U.S. Cl. CPC G01N 33/54373; G01N 15/1434; G01N 2015/0065

(57) ABSTRACT

Disclosed herein is a biomolecular detection device (1) for analyzing a cell, vesicle or a cellular or vesicular component, comprising an evanescent illuminator with an optical coupling unit configured for generating an evanescent field from coherent light (L) with a predefined wavelength on a first surface of the evanescent illuminator. The first surface of evanescent illuminator comprises a template nanopattern (5), containing a coherent arrangement of a plurality of predetermined lines along which membrane recognition elements for a binder structure (82) of a transmembrane protein (81), preferably a laterally diffusible transmembrane protein, of the cell, vesicle or the cellular or vesicular component (8) are arranged. The membrane recognition elements (53) are configured to bind the binder structure (82) of the transmembrane protein (81) for forming a transmembrane nanopattern within the cell, vesicle or the cellular or vesicular component (8) based on the template nanopattern (5) of the evanescent illuminator, such that light of the evanescent field is scattered by the cell, vesicle or the cellular or vesicular component (8) bound to the membrane recognition elements (53). The predetermined lines are arranged such that light scattered by the cell, vesicle or cellular or vesicular components (8) bound to the membrane recognition elements (53) constructively interferes at a predefined detection site (7) with a difference in optical path length that is an integer multiple of the predefined wavelength of the coherent light (L).

### CROSS-REFERENCE TO RELATED APPLICATIONS

This application is the United States national phase of International Application No. PCT/EP2020/075914 filed Sep. 16, 2020, and claims priority to European Patent Application No. 19197858.4 filed Sep. 17, 2019, the disclosures of each of which are hereby incorporated by reference in their entireties.

### BACKGROUND

### Field of Disclosure

The present invention lies in the field of biomolecular detection devices and their use for detecting biomolecular interactions, in particular intracellular or intravesicular interactions. The invention further comprises a method for generating a transmembrane nanopattern within a cell, vesicle, artificial or cellular component.

### Background

Detection devices are used, for example, as biosensors in a large variety of applications. One particular application is the detection or monitoring of binding affinities or processes. For example, with the aid of such biosensors various assays detecting the binding of target samples to binding sites can be performed. Typically, large numbers of such assays are performed on a biosensor at spots which are arranged in a two-dimensional microarray on the surface of the biosensor. The use of microarrays provides a tool for the simultaneous detection of the binding affinities or processes of different target samples in high-throughput screenings. For detecting the affinities of target samples to bind to specific binding sites, the affinity of target molecules to bind to specific capture molecules, a large number of capture molecules are immobilized on the outer surface of the biosensor at individual spots (e.g. by ink-jet spotting or photolithography). Each spot forms an individual measurement zone for a predetermined type of capture molecule. The binding of a target molecule to a specific type of capture molecule is detected and is used to provide information on the binding affinity of the target molecule with respect to the specific capture molecule.

A known technique for detecting binding affinities of target samples utilizes fluorescent labels. The fluorescent labels are capable of emitting fluorescent light upon excitation. The emitted fluorescent light has a characteristic emission spectrum which identifies the present fluorescent label at a particular spot. The identified fluorescent label indicates that the labelled target molecule has bound to the particular type of binding sites present at this spot.

A sensor for detecting labelled target samples is described in the article “Zeptosens' protein microarrays: A novel high performance microarray platform for low abundance protein analysis”, Proteomics 2002, 2, S. 383-393, Wiley-VCH Verlag GmbH, 69451 Weinheim, Germany. The sensor described therein comprises a planar waveguide arranged on a substrate. The planar waveguide has an outer surface capable of attaching a plurality of binding sites thereon. Moreover, the planar waveguide has a plurality of incoupling lines for coupling a beam of coherent light into the planar waveguide in a manner such that a beam of coherent light propagates along the planar waveguide. The coherent light propagates through the planar waveguide under total internal reflection with an evanescent field of the coherent light propagating along the outer surface of the planar waveguide. The depth of penetration of the evanescent field into the medium of lower refractive index at the outer surface of the planar waveguide is in the order of magnitude of a fraction of the wavelength of the coherent light propagating through the planar waveguide. The evanescent field excites the fluorescent labels of the labelled target samples bound to the binding sites arranged on the surface of the planar waveguide. Due to the very small depth of penetration of the evanescent field into the optically thinner medium at the outer surface of the planar waveguide, only the labelled samples bound to the binding sites immobilized on the outer surface of the planar waveguide are excited. The fluorescent light emitted by these labels is then detected with the aid of a CCD camera.

While it is principally possible to detect the binding affinities using fluorescent labels, this technique is disadvantageous in that the detected signal is produced by the fluorescent labels rather than by the binding partners themselves. In addition, labelling the target samples requires additional preparation steps. Moreover, labelled target samples are comparatively expensive. Another disadvantage is the falsification of the results caused by steric hindrance of the fluorescent labels at the target sample which might interfere with the binding of the target samples to the capture molecules. Further disadvantages are the falsification of the results due to photobleaching of the labels or quenching effects. In addition, fluorescent labelling may significantly influence the chemical, biological, pharmacological and physical properties of the compound of interest. Thus, measurements relying solely on fluorescent labelling may be falsified by the presence of such a label.

### SUMMARY

A crucial requirement for analyzing biological samples is the discrimination between specific binding of a compound or structural moiety of interest to a binding site from non-specific binding. Known strategies for addressing this issue such as surface plasmon resonance (SPR) or Mach Zehnder rely strongly on reference measurements and are only suitable for measurements under static conditions. Thus, such techniques are typically not suitable for measurements of highly complex environments, such as detection of biomolecular interactions within a living cell. SPR measures the refractive index change upon receptor-ligand binding in the vicinity of the sensor surface. This technique however has the disadvantage that it is susceptible to any refractive index change in the entire volume of the evanescent field in the vicinity of the surface. Id est, such sensors simply integrate the refractive index in the sensing volume and cannot discriminate its different spatial frequency components. Therefore, non-specific binding to the sensor surface as well as temperature gradients and the different buffer composition still pose a significant problem.

As G-protein coupled receptors (GPCRs) have evolved as particular prominent targets for drug candidates, due to their involvement in the development and progression of many diseases such as pain, asthma, inflammation, obesity and cancer, detailed analysis of these receptors in living cells are highly desirable. Whole cell assays to determine GPCR activity traditionally rely on the detection of distinct intracellular second messengers (cAMP, Ca²⁺), relocalization of fluorescently tagged proteins (arrestin recruitment to the receptor or receptor internalization) or the expression of a reporter gene under control of a GPCR-activated signaling cascade. However, as pointed out above, fluorescent labelling requires considerable biomolecular modifications, such as the overexpression of proteins or introduction of fluorescent labels, which are not always possible or desirable as these can alter cellular physiology or drug pharmacology. Furthermore, GPCRs typically modulate more than one effector which can lead to cross sensitivity in these assays.

Label-free cellular assays, like the resonance waveguide grating biosensor, do not require any molecular labels. Generally, such refractometric sensors monitor the refractive index above the sensor chip by means of a propagating evanescent wave which defines the sensing volume. Redistribution of cellular content within this sensing volume results in an overall change in the refractive index, giving rise to the much-appreciated holistic picture of dynamic mass redistribution (DMR). In return, DMR is inherently cross sensitive, meaning different GPCRs mediated signaling pathways cannot be deconvolved spatiotemporally by the sensor.

It is therefore an overall object of the present invention to improve the state of the art regarding biomolecular detection devices, thereby preferably avoiding disadvantages of the prior art fully or partly.

In favorable embodiments, a biomolecular detection device is provided for detecting specific biomolecular interactions within a cell or a vesicle, preferably excluding any cross sensitivity.

In further favorable embodiments, a biomolecular detection device is provided for specifically monitoring transmembrane protein activity, preferably excluding any cross-sensitivity.

The overall objective is in a general way achieved by the subject-matter of the independent claims. Further advantageous and exemplary embodiments follow from the description and the figures.

According to a first aspect of the invention, the overall objective is achieved by a biomolecular detection device for analyzing a cell, vesicle or a cellular or vesicular component, preferably a living cell, comprising an evanescent illuminator with an optical coupling unit configured for generating an evanescent field from coherent light with a predefined wavelength on a first surface of the evanescent illuminator. The first surface of the evanescent illuminator comprises a template nanopattern, which contains a coherent arrangement of a plurality of predetermined lines along which membrane recognition elements for a binder structure of a transmembrane protein, preferably a laterally diffusible transmembrane protein, of the cell, vesicle or the cellular or vesicular component are arranged. The membrane recognition elements are configured to bind the binder structure of the transmembrane protein for forming a transmembrane nanopattern within the cell, vesicle or the cellular or vesicular component based on the template nanopattern of the evanescent illuminator, such that light of the evanescent field is scattered by the cell, vesicle or the cellular or vesicular component bound to the membrane recognition elements. The predetermined lines are arranged such that light scattered by the cell, vesicle or cellular or vesicular components bound to the membrane recognition elements constructively interferes at a predefined detection site with a difference in optical path length that is an integer multiple of the predefined wavelength of the coherent light. As the skilled person understands, the optical path length refers to the distance between a specific membrane recognition element and the predefined detection site. As the skilled person understands, the evanescent illuminator is an element which is able to generate an evanescent field from coherent light of a light source.

Typically, the cell may be a living cell. A cellular component may for example be only a part of the cell, in particular only a part of the cell membrane or a cellular compartment.

The plurality of predetermined lines is typically configured to act as a template of an optical grating having a plurality of grating lines, such as grooves, elongated protrusions, materials of periodical changes of refractive index and the like. Preferably, these lines are chemically predefined. Arranged along these lines are membrane recognition elements, which may serve as binding sites for a binder structure, in particular an extracellular binder structure of the cell, vesicle or cellular or vesicular component. Preferably, the membrane recognition elements are configured to specifically bind to the binder structure of the transmembrane protein of interest. Thus only the transmembrane protein of interest may be able to bind to the membrane recognition elements, while other proteins are not able to bind to the membrane recognition elements. The membrane recognition elements are typically covalently bound to the predetermined lines.

In some typical embodiments, predetermined lines are separated from each other by areas devoid of recognition elements. For example, the areas devoid of recognition elements may optionally contain a linker, which is bound to the first surface of the evanescent illuminator, particularly, a planar waveguide, wherein the linker comprises an end portion with a structural moiety that cannot bind to the binder structure of the transmembrane protein.

The template nanopattern allows for transferring the template nanopattern of the biomolecular detection device into a living cell, by selectively binding transmembrane proteins which are laterally diffusible within the cell, thus generating a transmembrane nanopattern in the cell itself. Typically, membrane recognition elements of multiple different predetermined lines bind to the same cell, i.e. more than one membrane recognition element binds to or is configured to bind to a single cell, vesicle or cellular or vesicular component. As a result, any biomolecular interaction, even intercellular or intracellular processes, that involve an interaction of the transmembrane protein can be selectively detected. Upon binding between the membrane recognition elements and the binder structure of the transmembrane protein, evanescent light is scattered and constructively interferes at a predefined detection site. The constructive interference of the scattered light relates to all bound transmembrane proteins and yields quadratic scaling of the measured intensity with respect to the number of transmembrane proteins. Importantly, random scattering of background molecules which are not bound to the molecular recognition elements interfere with equal probability both constructively and destructively. As a result, cross sensitivity is effectively suppressed. Surprisingly, even though living cells and also vesicles comprise highly uneven surfaces on the nanometer scale, hardly any cross sensitivity is observed due to any cell scattering, particularly membrane scattering. While measurements of living cells may be readily disturbed by signals overruling the light scattered at the bound membrane recognition elements, no significant loss in sensitivity has been observed. This is surprising, as the cells, vesicles or cellular components are significantly larger than the distances between the predetermined lines of the nanopattern. Thus, in general in the embodiments disclosed herein, the membrane recognition elements of multiple different predetermined lines bind to the same cell. Typically, the distance between two directly adjacent predetermined lines is between 2 to 100 times smaller, preferably 10 to 50 times smaller, as the single cell.

In some embodiments, the evanescent illuminator comprises or is a carrier with a planar waveguide arranged on a surface of the carrier and an optical coupler as the optical coupling unit for coupling coherent light of a predefined wavelength into the waveguide such that the coherent light propagates through the planar waveguide with an evanescent field of the coherent light propagating along a first surface of the planar waveguide and wherein the first surface of the planar waveguide comprising the template nanopattern.

In some embodiments, the evanescent illuminator is a total internal reflection system configured for providing a beam of coherent light at the predetermined wavelength and at a predetermined angle onto the first surface of the evanescent illuminator by means of the optical coupling unit, particularly by a prism or any other suitable optical element.

Typically, the coherent light source that powers the evanescent illuminator and/or the coherent light propagating along the planar waveguide has a predetermined wavelength and is preferably monochromatic. Usually, visible or near infrared light may be used. The coherent evanescent field is scattered by scattering centers formed by the cells, vesicles or cellular components bound to the membrane recognition elements which are arranged on the different predetermined lines. The scattered intensity at any location can be determined by first adding the electric field contributions from each of the individual scattering centers and squaring the resulting phasor. A maximum of the scattered intensity, when transmembrane components are arranged to the nanopattern, is located at the predetermined detection location because the predetermined lines are arranged such that at the predetermined detection location, the optical path length of the light scattered by the different scattering centers differs by an integer multiple of the wavelength of the light. Thus, the light scattered by the cells, vesicles or cellular components bound to the membrane recognition elements interferes constructively at a predetermined detection location. The requirement of constructive interference is met by any scattered light originating from the location of the predetermined lines. The intensity pattern at the predetermined detection location preferentially forms but is not limited to a diffraction limited Airy disk. In essence, any shape accessible by Fourieroptics is possible. For any shape, the signal is best recoverable with a matched filter.

The scattering of an isolated membrane recognition element-transmembrane complex with refractive index n_{R} embedded in a medium with refractive index n₀ in plane-polarized light is:

$$\frac{{I}_{scat} ( r )}{{I}_{R}} = \frac{9 {π}^{2} {n}_{0}^{4}}{{r}^{2} {λ}^{4}} \vert \frac{{n}_{R}^{2} - {n}_{0}^{2}}{{n}_{R}^{2} + 2 {n}_{0}^{2}} \vert {V}_{R}^{2} = \frac{9 {π}^{2} {n}_{0}^{4}}{{r}^{2} {λ}^{4}} {( \frac{dn}{d c} )}^{2} \frac{{M}_{R}^{2}}{{N}_{A}^{2}} \overline{n} ( {ρ}_{R} ) ,$$

wherein

r is the distance from the compound of interest to the detection site

λ is the vacuum wavelength

I_{R} is the intensity of the plane-polarized field incident on the transmembrane complex

V_{R} the volume of the transmembrane complex;

and wherein

$${V}_{R} = \frac{dn}{d c} \frac{{M}_{R}}{{N}_{A} ( {n}_{R} - {n}_{0} )} ; \overline{n} ( {ρ}_{R} ) = \frac{{( {n}_{R} + {n}_{0} )}^{2}}{{( {n}_{R}^{2} + 2 {n}_{0}^{2} )}^{2}} ,$$

M_{R} is the molar mass of the transmembrane complex

N_{A} is the Avogadro constant

$$\frac{dn}{d c}$$

is the refractive index increment for proteins in water.

Thus, the intensity of the scattered light provides access to the molecular mass increase occurring during a biomolecular interaction in which the transmembrane protein is involved. For example, if a certain messenger compound binds to the transmembrane protein, the biomolecular detection device allows for the calculation of the mass increase. Furthermore, due to the mass increase, even dimerization of a receptor can be observed without any cross-sensitivity.

In the case of the evanescent illuminator comprising of a planar waveguide, the planar waveguide typically has a thickness in the range of a few hundred nanometers, i.e. of less than 1 mm, particularly less than 300 nm, preferably between 100 and 200 nm. The evanescent illuminator according to any of the embodiments herein usually has a thickness of up to 1 cm, in particular up to 0.8 mm for stabilizing the waveguide. The evanescent field decreases exponentially and typically reaches at least the membrane of a cell, vesicle or cellular or vesicular component. For example, the evanescent field may penetrate up to 80 to 100 nm of the cell, vesicle or cellular or vesicular component. The penetration depth can in principle be tailored to the application by adjusting the waveguide parameters or wavelength in embodiments that include planar waveguides and adjusting the wavelength and the angle of total internal reflection in the case of embodiments of total internal reflection.

In further embodiments, the membrane recognition element is an antibody being specific to at least the binder structure of the transmembrane protein. Alternatively, the membrane recognition element contains an electrophile moiety for establishing a covalent bond with the binder structure of the transmembrane protein, in particular a guanine or cytosine derivative. Alternatively, a nucleophile moiety may be employed.

In some embodiments, the plurality of predetermined lines comprises curved lines with a curvature configured such that light of the evanescent field scattered by the cell, vesicle or the cellular or vesicular component bound to the membrane recognition elements interferes at the predefined detection site.

Preferably, the overall shape of the template nanopattern may be round, particularly circular. In embodiments involving planar waveguides it preferentially may be square to avoid lensing effects of the guided mode.

In preferred embodiments, the curved lines are arranged with a decreasing distance between adjacent lines in the propagation direction of the light for focusing the light scattered by the cell, vesicle or cellular or vesicular component into the predetermined detection site. Alternatively, the plurality of predetermined lines may comprise straight lines being arranged with a predefined angle to the propagation direction of the light such that the diffracted light is coupled to another waveguide mode (Bragg-condition). An additional physical out-coupler may be employed for focusing the diffracted light into the predefined detection site. Alternatively, the plurality of predetermined lines may comprise straight or almost straight lines being arranged with a predefined angle to the propagation direction of the light such that the diffracted light forms a freely propagating beam that is focused by a lens or a lens system of the detector. In further embodiments, the distance between adjacent lines of the predetermined lines may be less than 1.5 μm, preferably less than 1 μm.

In certain embodiments, the coherent arrangement of predefined lines comprises a circular contour, wherein the circular contour has a diameter of up to 1000 μm, preferably up to 500 μm.

In some embodiments the number of recognition elements is up to 10¹⁰ per mm², preferably 10⁷ to 10¹⁰ per mm².

In further embodiments the first surface of the evanescent illuminator comprises a cell adhesive. The cell adhesive may for example comprise molecules with integrin-binding peptides, such as RGD, YIGSR, IKVAV, etc., or other cell binding molecules, such as chondroitin sulfate, hyaluronic acid, etc. Alternatively, or additionally, the cell adhesive may contain positively charged polymers, such as poly-lysine and/or natural cell adhesives, such as fibronectin, collagen, vitronectin, laminin or other suitable cell adhesives.

In some embodiments at least one cell, vesicle or cellular or vesicular component is bound via preferably an extracellular or extravesicular binder structure of a transmembrane protein to the membrane recognition elements.

In certain embodiments, the distance between adjacent predetermined lines decreases in the direction of propagation of the light of the evanescent field.

In further embodiments, the plurality of predetermined lines are arranged on the outer surface of the evanescent illuminator in a manner such that their locations in xⱼ,yⱼ coordinates are geometrically defined by the equation:

$${x}_{j} = \frac{λ N ( {A}_{0} + j ) - \sqrt{{n}_{S}^{2} ( {N}^{2} - {n}_{S}^{2} ) ( {y}_{j}^{2} + {f}^{2} ) + {( {n}_{S} λ )}^{2} {( {A}_{0} + j )}^{2}}}{{N}^{2} - {n}_{S}^{2}}$$

wherein
λ is the vacuum wavelength of the propagating light;
N is the effective refractive index of the guided mode in the planar waveguide; N depends on the thickness of the planar waveguide, the refractive index of the carrier, the refractive index of a medium on the first surface of the planar waveguide and the polarization of the guided mode; In the case of total internal reflection systems, N can be expressed via the angle of total internal reflection θₘ: N=nₛ sin(θ_{TIR}).
n_{S} is the refractive index of the carrier
f is the focal distance of the predetermined detection location;
A₀ is an integer which is chosen to be close to the product refractive index n_{S} and the focal distance of the predetermined detection location f of the carrier divided by the wavelength λ; and
j is a running integer indicating the index of the respective line.

The chosen integer A₀ assigns negative x-values at the center of the lines with negative j values and positive x-values at the center of lines with positive j values. Or to say it in other words, the integer A₀ defines the origin of the x,y coordinates frame that is used for the location of the lines at the outer surface of the evanescent illuminator; the chosen A₀ value puts the detection location at x=0, y=0, z=−f.

In some embodiments the planar waveguide has a refractive index n_{w} which is substantially higher than the refractive index nₛ of the carrier and which is also substantially higher than the refractive index n_{med} of the medium on the first surface of the planar waveguide. The predetermined wavelength of the light the evanescent field may in general have a penetration depth in the range of 40 nm to 200 nm. The term “substantially higher” shall be understood as designating a difference in refractive index allowing a coupling in of the light into the planar waveguide where it propagates under total internal reflection. The light propagating along the planar waveguide has an evanescent field which propagates along the outer surface of the planar waveguide. The evanescent field has a penetration depth which depends on the index n_{med}, the parameter N (in case of the planar waveguide this is the effective refractive index), as well as on the wavelength of the propagating light, so that the penetration depth can be adapted such that the light of the evanescent field is coherently scattered by the cell, vesicle or cellular or vesicular component bound to the membrane recognition elements located on or in proximity to the predetermined lines on the outer surface. The approximate values of the penetration depth mentioned above are to be understood to explicitly include the exact boundary values thereof.

In further embodiments of planar waveguides, the distance between the optical coupler and the template nanopattern may be in the range of several centimeters to 1 mm, such as for example 5 cm to 1 mm.

In certain embodiments, the carrier may be made of glass or a transparent polymer. An exemplary material of the waveguide is a transition metal oxide, such as tantalumpentoxide.

In some embodiments, the optical coupler may comprise a plurality of grating lines, in particular straight grating lines, being perpendicular to the propagation direction of the light for allowing coherent coupling of the coherent light under a predefined angle into the planar waveguide.

In certain embodiments, the biomolecular detection device may comprise more than one template nanopattern. For example, the device may comprise more than one waveguide, each comprising a single nanopattern or one or more waveguides each may comprise one or more template nanopatterns, which may optionally be superimposed on top of each other.

In some embodiments, the predetermined lines are separated from each other by areas devoid of membrane recognition elements. These areas are configured to inverse an optical modulation, which is induced by the binding of a structural recognition element to the binder structure of the transmembrane protein, such that the signal obtained from binding of the structural recognition element to the binder structure of the transmembrane protein is provided in a different operating window of the biomolecular detection device as compared to the signal obtained from binding of the structural recognition element to the binder structure of the transmembrane protein of a biomolecular detection device without areas devoid of membrane recognition elements, configured to inverse the optical modulation. Preferably, the signal obtained from binding of the structural recognition element to the binder structure of the transmembrane protein is close to zero, preferably essentially zero. Thus, the signal obtained from binding of the structural recognition element to the binder structure of the transmembrane protein is canceled out by the areas devoid of recognition elements. As the signal to noise ratio is optimal at a measured spot intensity of essentially zero, such a biomolecular detection device allows for a better detection of intracellular or intravesicular interactions, such as binding of a drug or a messenger molecule to a receptor.

In certain embodiments, the areas devoid of recognition elements comprise a biasing mass configured for inversing the optical modulation, which is induced by the binding of a structural recognition element to the binder structure of the transmembrane protein. Typically, the biasing mass matches the mass of the transmembrane protein, which is configured to be bound to the structural recognition elements of the biomolecular detection device.

In some embodiments, the biasing mass is a non-absorbing nanoparticle or a polymer molecule such as a protein.

According to a further aspect of the invention, the overall objective is achieved by the use of a biomolecular device according to any of the embodiments described herein for detecting molecular interactions associated with cells, vesicles or cellular components. In particular, the use may be a method for detecting biomolecular interactions.

In some embodiments, the use includes: providing a biomolecular detection device according to any of the embodiments described herein; applying a cell or a vesicle to the membrane recognition elements, wherein the cell or the vesicle comprises a membrane and at least one transmembrane protein with an extracellular or extravesicular binder structure; optionally laterally diffusing at least one transmembrane protein along the membrane; aligning at least one transmembrane protein of the cell or the vesicle according to the template nanopattern of the first surface of the evanescent illuminator, such that a transmembrane nanopattern is formed in the membrane of the cell or the vesicle, wherein the transmembrane pattern corresponds at least partially to the template nanopattern of the first surface of the evanescent illuminator; generating a beam of coherent light at a predefined beam generation location relative to the plurality of predetermined lines, the beam of coherent light having a predefined wavelength and being incident on the membrane recognition elements with the bound transmembrane protein in a manner that diffracted portions of the incident beam of coherent light constructively interfere at the predefined detection site relative to the plurality of predetermined lines with a difference in optical path length that is an integer multiple of the predefined wavelength of the coherent light to provide a signal representative of the membrane recognition elements with the transmembrane protein of a cell, vesicle or cellular or vesicular component bound thereto at the predefined detection site; measuring the signal representative for the membrane recognition elements with the transmembrane of a cell, vesicle or cellular or vesicular component bound thereto.

Such embodiments consequently exploit the property of transmembrane proteins to be able to laterally diffuse within the cellular or vesicular membrane. Thus, as soon as the extracellular or extravesicular binding structure approaches a membrane recognition element of the biomolecular detection device, binding occurs and the transmembrane protein is locked in this position. This process is repeated with a plurality of transmembrane proteins, resulting in nanopattern within the membrane of the cell, vesicle or cellular or vesicular component, i.e. a transmembrane protein nanopattern or in general a transmembrane molecule nanopattern, which corresponds at least partially or fully to the template nanopattern of the biomolecular detection device.

In further embodiments, the use additionally comprises the step of comparing the measured signal representative of the membrane recognition elements with the transmembrane protein of a cell, vesicle or cellular or vesicular component bound thereto, with an unbound signal representative of only the membrane recognition elements. This step is preferred, but not crucial, as it may lead for a quantitative result, for example, when the mass of an interacting compound is to be determined.

In some embodiments, a signal obtained from areas devoid of membrane recognition elements inverses an optical modulation which is induced by the binding of a structural recognition element to the binder structure of the transmembrane protein, such that the signal obtained from binding of the structural recognition element to the binder structure of the transmembrane protein is provided in a different operating window of the biomolecular detection device, wherein preferably the different operating window is at an intensity close to zero. Thus, the signal obtained from binding of the structural recognition element to the binder structure of the transmembrane protein is canceled out by the areas devoid of recognition elements. As the signal to noise ratio is optimal at a measured spot intensity of essentially zero, such a biomolecular detection device allows for a better detection of intracellular or intravesicular interactions, such as binding of a drug or a messenger molecule to a receptor.

In further embodiments, the areas devoid of recognition elements comprise a biasing mass configured for inversing the optical modulation, which is induced by the binding of a structural recognition element to the binder structure of the transmembrane protein. Typically, the biasing mass matches the mass of the transmembrane protein, which is configured to be bound to the structural recognition elements of the biomolecular detection device. In some embodiments, the biasing mass is a nanoparticle or a polymer molecule such as a protein.

In certain embodiments, a cell is applied to the membrane recognition elements and wherein before generating the beam of coherent light and preferably after aligning the transmembrane protein and forming of the transmembrane nanopattern, the cell is modified such that only parts of the cell membrane remain on the biomolecular detection device

In some embodiments, a genetically modified cell or vesicle is applied to the membrane recognition elements, in particular, a cell or a vesicle which is modified such that it contains at least a binder structure of the transmembrane protein, which is configured to bind to the membrane recognition element of the biomolecular detection device. Such cells allow for using specifically designed transmembrane proteins, which may be specifically recognized by a corresponding membrane recognition element.

In further embodiments, the transmembrane protein may be engineered to contain an additional extracellular binding site or intracellular binding site, in addition to the binding site required to bind the transmembrane, e.g. via the transmembrane protein, to the membrane recognition elements on the predetermined lines. The additional binding site may for example be a HA (human influenza hemagglutinin), FLAG or 6His affinity tag or any moiety that allows binding of a molecular with a known molecular mass. That can be voluntarily expressed within the cells or added to the device. This has the advantage that the number of immobilized transmembrane proteins on the predetermined lines can be precisely quantified. Fluorescence imaging can be used to determine the number of the cells, which allows the average number of immobilized transmembrane proteins per cell to be quantified.

In further embodiments the binder structure of the transmembrane protein is specific to an antibody being arranged along the predefined lines of the evanescent illuminator of the biomolecular detection device.

In some embodiments the light beam of coherent light is generated only after a predefined binding time. For example, the binding time may be less than 4 h, in particular less than 2 h. In certain embodiments, the predefined binding time may be 0.5 to 4 h, particularly 1.5 to 3 h, after the cell, vesicle or cellular or vesicular component has been applied to the membrane recognition elements. It has been observed that after this time period, a large amount of the transmembrane proteins is bound to membrane recognition elements and the nanopattern is established within the membrane. Obviously, if the formation of the nanopattern is to be observed, the light beam of coherent light may be generated concomitantly or directly after application of the cell, vesicle or cellular or vesicular component.

In some embodiments the transmembrane protein comprises an intracellular or intravesicular fluorescent protein or a protein interacting with other intracellular elements. Thus, the biomolecular detection device may be combined with state of the art fluorescence spectroscopy. If the intracellular or intravesicular protein is configured for interacting with additional intracellular elements, whole intracellular signaling pathways can be observed, as the mass increase of the interaction of the additional cellular elements entails a change of intensity of the measured signal.

In further embodiments a protein of interest of the biomolecular interaction comprises a high-mass moiety. The use of such high-mass moieties is beneficial, as the higher molecular mass has a beneficial effect on the intensity of the obtained signal, thus even enabling single cell measurements. As the skilled person understands, a high-mass moiety typically has a significantly larger molecular weight than the rest of the transmembrane protein. For example, the high-mass moiety may have a molecular weight of 150 kDa or more.

In some embodiments additionally, particularly simultaneously, a fluorescent signal and/or bioluminescent and/or a refractometric signal is recorded. In the case of embodiments involving guided waves, in particular in a planar waveguide, a refractometric signal that constitutes the total mass change on the first surface of the evanescent illuminator can be determined from the shift of the predetermined detection location.

$$Δ Γ = \frac{1}{\frac{dn}{d c} f} \frac{{t}_{eff}}{2} \frac{N}{{n}_{c}} \frac{{n}_{f}^{2} - {n}_{c}^{2}}{{n}_{f}^{2} - {N}^{2}} Δ x$$

ΔΓ is the total mass density change on the surface of the evanescent illuminator

$$\frac{dn}{d c}$$

is the refractive index increment for proteins

f is the focal distance of the predetermined detection location

t_{eff} is the effective thickness of the guided mode

N is the effective refractive index of the guided mode

n_{c} is the refractive index of the cover medium

n_{f} is the refractive index of the waveguiding film

Δx is the shift of the predetermined detection location in the focal plane.

In further embodiments a single cell or a single vesicle is applied to the membrane recognition elements or a plurality of cells of vesicles is applied to the membrane recognition elements.

In some embodiments the cell density of the applied cells is 0.001 to 0.02 cells/μm², preferably 0.005 to 0.01 cells/μm². In other embodiments, the vesicle density if in the range of up to 200, preferably up to 100 vesicles/μm².

In further embodiments the signal of a molecular interaction representative for the membrane recognition elements with the transmembrane protein of a cell, vesicle or cellular or vesicular component bound thereto is associated with a direct or indirect molecular interaction of the transmembrane protein with an intercellular component.

In some embodiments the step of measuring the signal of a molecular interaction representative for the membrane recognition elements with the transmembrane of a cell, vesicle or cellular or vesicular component bound thereto at the predefined detection site is performed multiple times, in particular within regular predefined time intervals.

In further embodiments the signal obtained in at least some of the measurements is associated with a direct or indirect molecular interaction of the transmembrane protein with an intercellular component which is different from the molecular interaction measured during a previous measurement.

According to a further aspect of the invention, the overall objective is achieved by a method for generating a transmembrane nanopattern within a cell, vesicle or cellular or vesicular component, the method comprising: providing a biomolecular detection device according to any of the embodiments described herein, applying a cell or a vesicle is applied to the membrane recognition elements, wherein the cell or the vesicle comprises a membrane and at least one transmembrane protein with an extracellular or extravesicular binder structure, optionally laterally diffusing at least one transmembrane protein along the membrane, binding the extracellular binding structure of the at least one transmembrane protein to any of the membrane recognition elements and aligning the at least one transmembrane protein according to the template nanopattern of the first surface of the evanescent illuminator, such that a transmembrane nanopattern is formed in the membrane of the cell or the vesicle, wherein the transmembrane pattern corresponds at least partially or fully to the template nanopattern of the first surface of the evanescent illuminator.

In some embodiments a genetically modified cell or vesicle is applied to the membrane recognition elements, in particular, a cell or a vesicle which is modified such that it contains at least a binder structure of the transmembrane protein, which is configured to bind to the membrane recognition element of the biomolecular detection device.

In further embodiments the time between applying the cell, vesicle or cellular or vesicular component until formation of the transmembrane nanopattern in the membrane ranges from 0.5 to 4 h, preferably) 0.5 to 3 h.

In some embodiments the extracellular binder structure of the transmembrane protein is specific to an antibody being arranged along the predefined lines of the evanescent illuminator of the biomolecular detection device. Preferably, the antibodies are configured to recognize an affinity tag of the transmembrane protein. The affinity tag may for example be a HA (human influenza hemagglutinin), FLAG or 6His affinity tag.

In some embodiments the transmembrane protein comprises an intracellular or intravesicular fluorescent protein or a protein configured for interacting with other intracellular elements. Preferably, the protein is configured for specifically interacting with other intracellular elements.

In some embodiments a single cell or a single vesicle is applied to the membrane recognition elements or wherein a plurality of cells of vesicles is applied to the membrane recognition elements.

In further embodiments the cell density of the applied cells is 0.001 to 0.02 cells/μm², preferably 0.005 to 0.01 cells/μm².

The invention is further described by the following clauses:

Clause 1: Biomolecular detection device (1) for analyzing a cell, vesicle or a cellular or vesicular component, comprising a evanescent illuminator with an optical coupling unit configured for generating an evanescent field from coherent light with a predefined wavelength on a first surface of the evanescent illuminator, the first surface of the evanescent illuminator comprising a template nanopattern (5), containing a coherent arrangement of a plurality of predetermined lines along which membrane recognition elements for a binder structure (82) of a transmembrane protein (81), preferably a laterally diffusible transmembrane protein, of the cell, vesicle or the cellular or vesicular component (8) are arranged, wherein the membrane recognition elements (53) are configured to bind the binder structure (82) of the transmembrane protein (81) for forming a transmembrane nanopattern within the cell, vesicle or the cellular or vesicular component (8) based on the template nanopattern (5) of the evanescent illuminator (3), such that light of the evanescent field is scattered by the cell, vesicle or the cellular or vesicular component (8) bound to the membrane recognition elements (53), and wherein the predetermined lines are arranged such that light scattered by the cell, vesicle or cellular or vesicular components (8) bound to the membrane recognition elements (53) constructively interferes at a predefined detection site (7) with a difference in optical path length that is an integer multiple of the predefined wavelength of the coherent light (L).

Clause 2: Biomolecular detection device (1) according to clause 1, wherein the evanescent illuminator comprises a carrier (2) with a planar waveguide (3) arranged on a surface of the carrier and an optical coupler (4) as the optical coupling unit for coupling coherent light (L) of a predefined wavelength into the waveguide (3) such that the coherent light propagates through the planar waveguide (3) with an evanescent field of the coherent light (L) propagating along a first surface of the planar waveguide (3) and wherein the first surface of the planar waveguide comprising the template nanopattern (5).

Clause 3: Biomolecular detection device (1) according to clause 1, wherein the evanescent illuminator is a total internal reflection system (□) configured for providing a beam of coherent light (L) at the predetermined wavelength and at a predetermined angle onto the first surface of the evanescent illuminator by means of the optical coupling unit, particularly by a prism.

Clause 4: Biomolecular detection device (1) according to any of the previous clauses, wherein the membrane recognition elements (53) are antibodies being specific to at least the binder structure (82) of the transmembrane protein (81), or wherein the membrane recognition elements (53) contain an electrophile moiety for establishing a covalent bond with the binder structure (82) of the transmembrane protein (81), in particular a guanine or cytosine derivative.

Clause 5: Biomolecular detection device according to any of the preceding clauses, wherein the plurality of predetermined lines comprises curved lines with a curvature configured such that light of the evanescent field scattered by the cell, vesicle or the cellular or vesicular component bound to the membrane recognition elements interferes at the predefined detection site.

Clause 6: Biomolecular detection device according to clause 3, wherein the coherent arrangement of predefined lines comprises a circular contour, wherein the circular contour has a diameter of up to 1000 μm, preferably up to 500 μm.

Clause 7: Biomolecular detection device according to any of the previous clauses, wherein the number of recognition elements is up to 10¹⁰ per mm².

Clause 8: Biomolecular detection device according to any of the previous clauses, wherein the first surface of the evanescent illuminator comprises a cell adhesive.

Clause 9: Biomolecular detection device according to any of the previous clauses, wherein at least one cell, vesicle or cellular or vesicular component is bound via the binder structure of the transmembrane protein to the membrane recognition elements.

Clause 10: Biomolecular detection device according to any of the previous clauses, wherein the predetermined lines are separated from each other by areas devoid of membrane recognition elements and wherein the area devoid of membrane recognition elements are configured to inverse an optical modulation, which is induced by the binding of a structural recognition element to the binder structure of the transmembrane protein, such that the signal obtained from binding of the structural recognition element to the binder structure of the transmembrane protein is provided in a different operating window of the biomolecular detection device, wherein the different operating window is at an intensity close to zero.

Clause 11: Biomolecular detection device according to clause 8, wherein the areas devoid of recognition elements comprise a biasing mass configured for inversing the optical modulation.

Clause 12: Use of a biomolecular detection device according to any of clauses 1 to 9 for detecting molecular interactions associated with cells, vesicles or cellular components.

Clause 13: The use according to clause 10, wherein:
A biomolecular detection device according to any of clauses 1 to 11 is provided;
A cell or a vesicle is applied to the membrane recognition elements, wherein the cell or the vesicle comprises a membrane and at least one transmembrane protein with an extracellular or extravesicular binder structure;
Optionally at least one transmembrane protein is laterally diffused along the membrane;
At least one transmembrane protein of the cell or the vesicle is aligned according to the template nanopattern of the first surface of the evanescent illuminator, such that a transmembrane nanopattern is formed in the membrane of the cell or the vesicle, wherein the transmembrane pattern corresponds at least partially to the template nanopattern of the first surface of the evanescent illuminator;
Generating a beam of coherent light at a predefined beam generation location relative to the plurality of predetermined lines, the beam of coherent light having a predefined wavelength and being incident on the membrane recognition elements with the bound transmembrane protein in a manner that diffracted portions of the incident beam of coherent light constructively interfere at the predefined detection site relative to the plurality of predetermined lines with a difference in optical path length that is an integer multiple of the predefined wavelength of the coherent light to provide a signal representative of the membrane recognition elements with the transmembrane protein of a cell, vesicle or cellular or vesicular component bound thereto at the predefined detection site;
Measuring the signal representative for the membrane recognition elements with the transmembrane of a cell, vesicle or cellular or vesicular component bound thereto.

Clause 14: The use according to clause 11, further comprising the step of comparing the measured signal representative of the membrane recognition elements with the transmembrane of a cell, vesicle or cellular or vesicular component bound thereto, with an unbound signal representative of only the membrane recognition elements.

Clause 15: The use according to any of clauses 10 to 12, wherein a cell is applied to the membrane recognition elements and wherein before generating the beam of coherent light and preferably after aligning the transmembrane protein and forming of the transmembrane nanopattern, the cell is modified such that only parts of the cell membrane remain on the biomolecular detection device.

Clause 16: The use according to any of clauses 10 to 13, wherein a genetically modified cell or vesicle is applied to the membrane recognition elements, in particular, a cell or a vesicle which is modified such that it contains at least a binder structure of the transmembrane protein, which is configured to bind to the membrane recognition element of the biomolecular detection device.

Clause 17: The use according to any of clauses 10 to 14 wherein the binder structure of the transmembrane protein is specific to an antibody being arranged along the predefined lines of the evanescent illuminator of the biomolecular detection device.

Clause 18: The use according to any of clauses 10 to 15, wherein generating the light beam of coherent light is only performed after a predefined binding time.

Clause 19: The use according to any of clauses 10 to 16, wherein the transmembrane protein comprises an intracellular or intravesicular fluorescent protein or a protein interacting with other intracellular elements.

Clause 20: The use according to any of clauses 10 to 17, wherein a protein of interest of the biomolecular interaction comprises a high-mass moiety.

Clause 21: The use according to any of clauses 10 to 18, wherein additionally, particularly simultaneously, a fluorescent and/or bioluminescent signal is recorded.

Clause 22: The use according to any of the clauses 10 to 19, wherein a single cell or a single vesicle is applied to the membrane recognition elements or wherein a plurality of cells of vesicles is applied to the membrane recognition elements.

Clause 23: The use according to any of clauses 10 to 20 wherein the cell density of the applied cells is 0.001 to 0.02 cells/μm², preferably 0.005 to 0.01 cells/μm².

Clause 24: The use according to any of clauses 10 to 21, wherein the signal of a molecular interaction representative for the membrane recognition elements with the transmembrane of a cell, vesicle or cellular or vesicular component bound thereto is associated with a direct or indirect molecular interaction of the transmembrane protein with an intercellular component.

Clause 25: The use according to any of the clauses 10 to 22, wherein the step of measuring the signal of a molecular interaction representative for the membrane recognition elements with the transmembrane of a cell, vesicle or cellular or vesicular component bound thereto at the predefined detection site is performed multiple times, in particular within regular predefined time intervals.

Clause 26: The use according to clause 23, wherein the signal obtained in at least some of the measurements is associated with a direct or indirect molecular interaction of the transmembrane protein with an intercellular component which is different from the molecular interaction measured during a previous measurement.

Clause 27: The use according to any of clauses 10 to 24, wherein the transmembrane protein is engineered to contain an additional extracellular binding site or intracellular binding site, in addition to the binding site required to bind the transmembrane to the membrane recognition elements on the predetermined lines.

Clause 28: Method for generating a transmembrane nanopattern within a cell, vesicle or cellular or vesicular component, the method comprising:
Providing a biomolecular detection device according to any of clauses 1 to 11;
Applying a cell or a vesicle to the membrane recognition elements, wherein the cell or the vesicle comprises a membrane and at least one transmembrane protein with an extracellular or extravesicular binder structure;
Optionally laterally diffusing at least one transmembrane protein along the membrane;
Binding the extracellular binding structure of the at least one transmembrane protein to any of the membrane recognition elements and aligning the at least one transmembrane protein according to the template nanopattern of the first surface of the evanescent illuminator, such that a transmembrane nanopattern is formed in the membrane of the cell or the vesicle, wherein the transmembrane pattern corresponds at least partially to the template nanopattern of the first surface of the evanescent illuminator.

Clause 29: The method according to clause 26, wherein a genetically modified cell or vesicle is applied to the membrane recognition elements, in particular, a cell or a vesicle which is modified such that it contains at least a binder structure of the transmembrane protein, which is configured to bind to the membrane recognition element of the biomolecular detection device.

Clause 30: The method according to any of clauses 26 or 27 wherein the time between applying the cell, vesicle or cellular or vesicular component until formation of the transmembrane nanopattern in the membrane ranges 0.5 to 4 h, preferably 1.5 to 3 h.

Clause 31: The method according to any of clauses 26 to 28, wherein the binder structure of the transmembrane protein is specific to an antibody being arranged along the predefined lines of the evanescent illuminator of the biomolecular detection device.

Clause 32: The method according to any of clauses 26 to 29, wherein the transmembrane protein comprises an intracellular or intravesicular fluorescent protein or a protein interacting with other intracellular elements.

Clause 33: The method according to any of the clauses 25 to 30, wherein a single cell or a single vesicle is applied to the membrane recognition elements or wherein a plurality of cells of vesicles is applied to the membrane recognition elements.

Clause 34: The method according to any of clauses 26 to 31, wherein the cell density of the applied cells is 0.001 to 0.02 cells/μm², preferably 0.005 to 0.01 cells/μm².

### BRIEF DESCRIPTION OF THE FIGURES

FIG. 1 shows a schematic representation of a biomolecular detection device according to a first embodiment of the invention;

FIG. 2 shows a schematic cross-sectional view of a biomolecular detection device according to another embodiment of the invention;

FIGS. 3a and 3b show a membrane of a cell and an enlarged view of a waveguide of a biomolecular detection device according to an embodiment of the invention;

FIGS. 4a to 4c show a signal obtained by a biomolecular detection device according to an embodiment of the invention during the generation of a nanopattern within a cell, vesicle or cellular or vesicular component.

FIGS. 5a and 5b show a signal obtained from applying HEK293 cells to a biomolecular detection device according to the invention and further stimulating and inhibiting β₂ARs.

FIGS. 6a and 6b show a signal obtained from applying HEK293 cells to a biomolecular detection device according to the invention and further applying an off-target stimulant (FIG. 6a) as well as a positive control with an on-target stimulant (FIG. 6b).

FIGS. 7a and 7b show a schematic representation of a biomolecular detection device according to another embodiment of the invention.

FIGS. 8a and 8b show a schematic representation of a biomolecular detection device according to another embodiment of the invention.

### DESCRIPTION

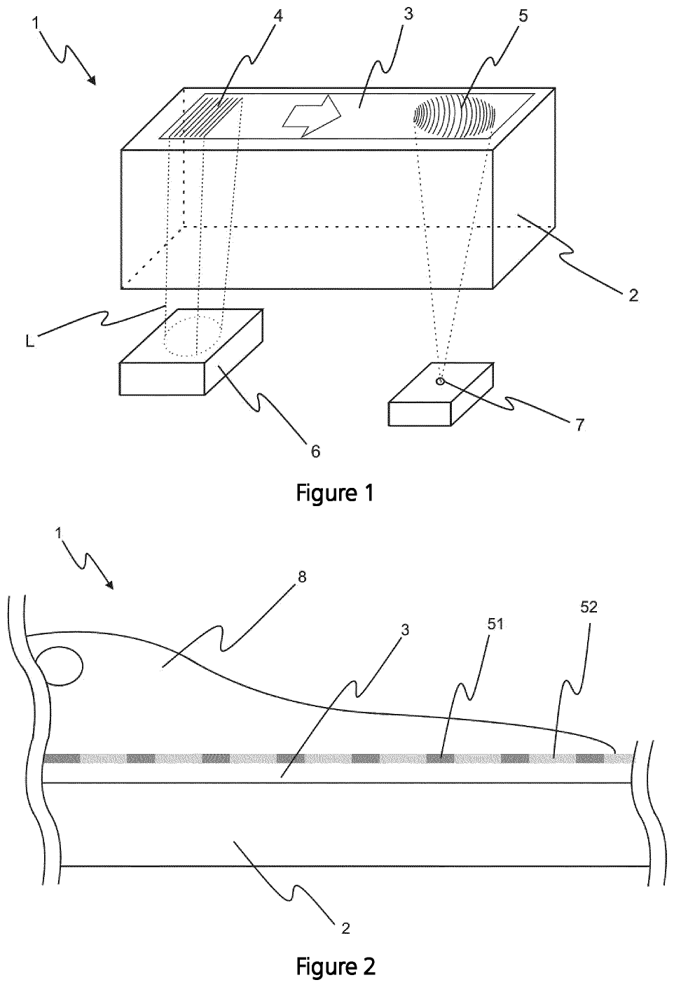

FIG. 1 shows a biomolecular detection device 1 according to an embodiment of the invention. The biomolecular detection device 1 comprises an evanescent illuminator comprising in this embodiment a carrier 2 with a surface on which planar waveguide 3 is arranged. The detection device further comprises optical coupler 4 for coupling coherent light L of a predefined wavelength into planar waveguide 3 such that coherent light propagates through the planar waveguide with an evanescent field of the coherent light propagating along a first surface of planar waveguide 3. The first surface of the planar waveguide is the surface facing away from carrier 2, i.e. the surface which is visible in FIG. 1. Besides optical coupler 4, the first surface of the waveguide 3 comprises template nanopattern 5, which contains a plurality of predetermined lines along which membrane recognition elements are arranged (not shown in FIG. 1). In general, a light source 6 is employed for providing an incoming beam of coherent light L towards optical coupler 4. Optical coupler 4 couples the coherent light L into planar waveguide 3 upon which coherent light L propagates in the direction of the arrow shown in FIG. 1 towards template nanopattern 5. If the membrane recognition elements are bound to a cell, vesicle or cellular or vesicular component, light is scattered and due to the predefined lines, which are arranged such that light scattered by the cell, vesicle or cellular or vesicular components bound to the membrane recognition elements constructively interferes at a predefined detection site 7. In the particular embodiment shown, the predetermined lines are curved lines having a curvature configured such that light of the evanescent field scattered by the cell, vesicle or the cellular or vesicular component bound to the membrane recognition elements interferes at the predefined detection site 7. The distance between each of the membrane recognition elements of template nanopattern 5 and detection site 7 is referred to as the optical path length.

FIG. 2 illustrates a schematic cross-section of a biomolecular detection device 1 shown in FIG. 1 through template nanopattern 5 along the propagation direction of the light L through planar waveguide 3. Device 1 contains carrier 2 with waveguide 3 arranged on its surface. Arranged on top of the first surface of planar waveguide 3 is living cell 8. Furthermore, template nanopattern 5 comprises ridges 51 and grooves 52. Ridges 51 are areas along which membrane recognition elements are arranged. Grooves 52 are areas which do not contain any membrane recognition elements. Thus, a binder structure of a transmembrane protein of cell 8 can only bind to nanopattern 5 at a corresponding ridge. It should be noted that the widths of the cell, nanopattern, waveguide and carrier do not provide any indication of their actual widths or the width ratios.

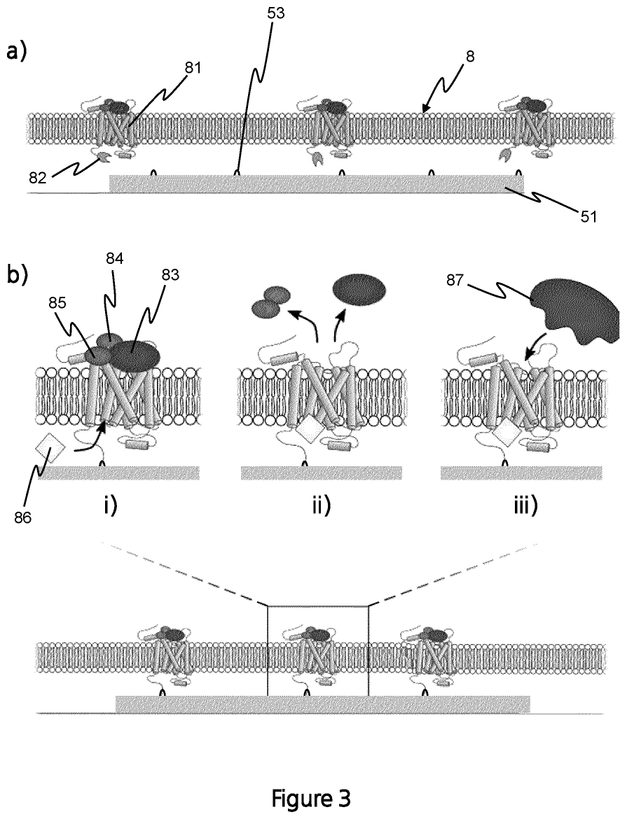

FIG. 3a shows an enlarged schematic representation of a biomolecular detection device of FIG. 2 directly after a cell 8 has been applied to the first surface of the waveguide. The cell comprises transmembrane proteins 81 with extracellular binder structure 82. In the particular embodiment shown, the transmembrane protein is a G-protein coupled receptor (GPCR). The template nanopattern of the waveguide comprises predetermined lines with ridge 51 comprising membrane recognition elements 53. As can be seen, binder structures 82 of the transmembrane proteins cannot yet interact with membrane recognition elements 53.

In the lower portion of FIG. 3b, the GPCRs were laterally diffused within the membrane, thereby enabling binding of the extracellular binder structure to the membrane recognition elements. As a result, each bound GPCR is locked at a specific position within the membrane. The overall arrangement of all GPCRs bound to membrane recognition elements is a nanopattern which corresponds to the template nanopattern of the waveguide. In other words, the template nanopattern of the waveguide has been transformed into the cell. The upper portion of FIG. 3b illustrates binding of ligand 86 to the extracellular portion of the nanopattern formed in the membrane (i). The attachment of the ligand entails a mass increase, which provides an increase of the measured signal. Binding of ligand 86 leads to an intracellular interaction. G-protein signaling is affected by ligand binding, in which the Ga subunit 83, Gβ subunit 84 and Gγ subunit 85 subunit are released. This release causes a mass decrease, which triggers a decrease of the observed signal (ii). Receptor desensitization requires recruitment of cytosolic protein 87, which again causes a mass increase and thus triggers an increase of the observed signal. Due to the absence of cross-sensitivity, direct information about the mass of the complex at a specific point in time can be obtained.

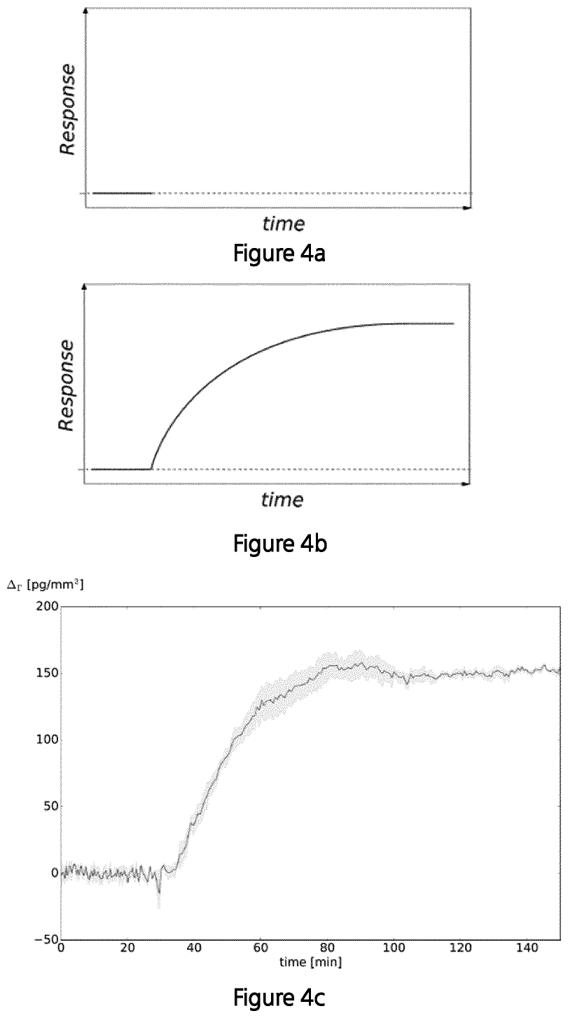

FIGS. 4a and 4b illustrate an ideal output signal obtained by using a biomolecular detection device as described herein. In FIG. 4a, the measurement has started and a constant response is obtained, as the binder structures of the transmembrane proteins are not yet bound to the membrane recognition elements of the waveguide. When binding occurs, the measured signal increases until it reaches a constant value (i.e. when all possible binder structures are bound to a membrane recognition element, see FIG. 4b). FIG. 4c shows an experimentally obtained signal obtained by binding β₂ARs (beta-2 adrenergic receptors) in HEK293 cells with a fused autoreactive SNAP tag protein to the molecular recognition elements of the nanopattern of the biomolecular detection device. In this particular embodiment, the SNAP tag protein is bound to a BG-NH₂ derivative which is bound to the waveguide. As can be seen, after around 80-100 min, a constant signal is observed.

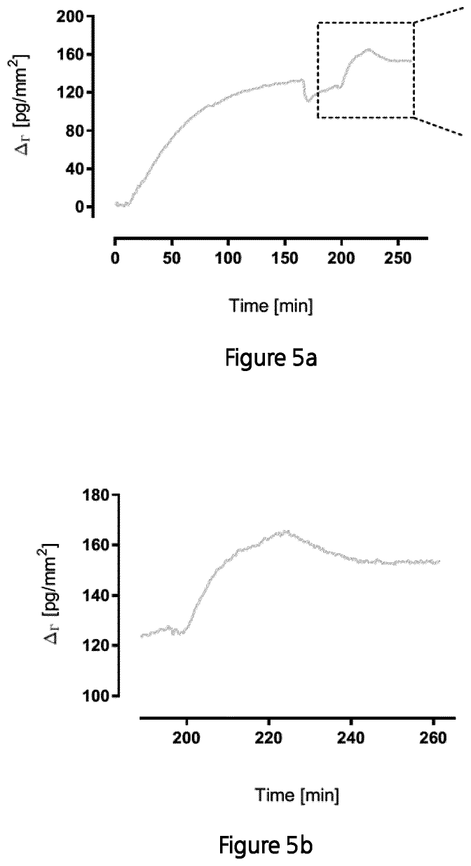

FIG. 5a shows the real time formation of a nanopattern and further the response obtained from stimulating and inhibiting the employed HEK293 cells. With reference to FIG. 5a, the first increase of the measured signal between 0 and 150 min corresponds to the formation of the nanopattern within the cell membrane. At 150 min, the cultivation has been changed to assay buffer resulting in a decrease of the signal. FIG. 5b shows an enlarged view of the dashed area in FIG. 5a. At around 200 min, the cells were stimulated with 1 μM isoproterenol, which resulted in an increase of the measured signal. Competitive inhibition of isoproterenol with 10 μM ICI 155.881 at around 225 min lead to a decrease of the signal and partial recovery of the initial state. This measurement proves that the obtained signal is a consequence of the observed intracellular interaction and not just the result of a free SNAP tag bound to the molecular recognition element.

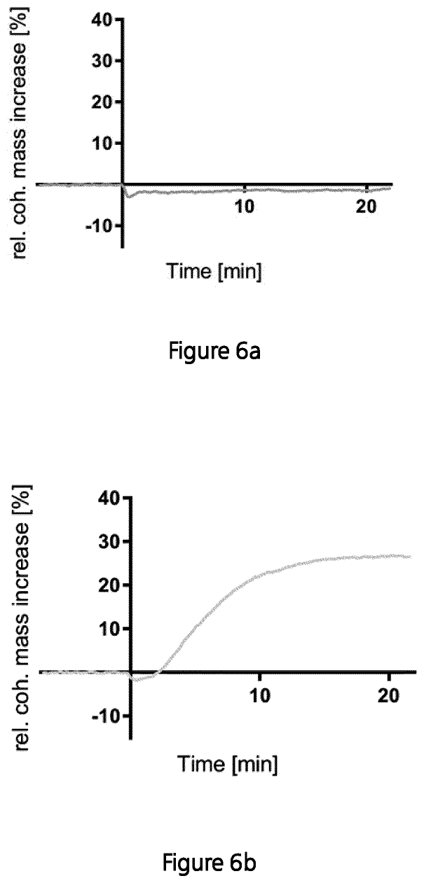

FIG. 6a shows the signal obtained upon stimulation of the HEK293 cells with 5 μM NECA to activate the off-target adenosine receptors. Due to slight variations in the initial measured signal intensity, the mass increase at the receptor, relative to the initial mass is depicted. However, with no contribution from off-target receptors this mass increase corresponds to the molecular weight increase relative to the molecular weight of the immobilized receptor complex (assuming all receptors were activated). The results show that no significant signal change is observed upon activation of the adenosine receptor with NECA. In contrast, a control experiment with 1 μM isoproterenol results in the signal increase which has already been shown in FIG. 4c. In summary, these results show that indeed no cross sensitivity of any off target interactions is observed and that these interactions do not influence the measurement result. As explained above, the reason for the absence of cross sensitivity is that the adenosine receptors are not bound to the molecular recognition elements and therefore no scattered light is received at the detection site. In fact, receptors that are distributed randomly, scatter light in an incoherent way and thus do not participate in this focusing effect. Similarly, morphological changes to the cell body are incoherent in their nature and therefore do not contribute to the measured signal either. This is in sharp contrast to DMR and other label-free assays that record both specific and nonspecific molecular interactions as well as morphological changes to the cell. As a consequence, cellular morphology can not only answer the question if GPCRs are activated but also shed light on the temporal occurrence and progression as well as mass of the molecules involved.

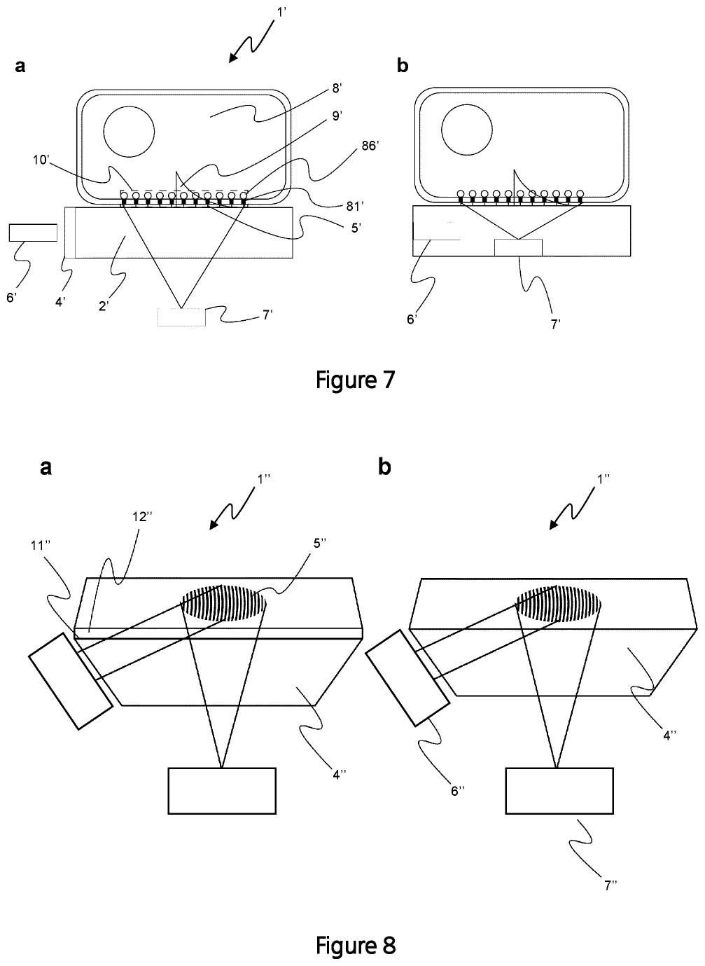

FIGS. 7a and 7b show a biomolecular detection device 1′ according to an embodiment of the invention. Device 1′ comprises an evanescent illuminator 2′ with an optical coupling unit 4′ configured for generating an evanescent field 9′ from coherent light with a predefined wavelength of a light source 6′ on a first surface of the evanescent illuminator 2′. The first surface of the evanescent illuminator 2′ comprises template nanopattern 5′ containing a coherent arrangement of a plurality of predetermined lines along which membrane recognition elements for a binder structure of a transmembrane protein 81 of cell 8′. As can be seen, by establishing a chemical bond between the membrane recognition element and the laterally diffusible transmembrane proteins 81, the nanopattern 5′ of the evanescent illuminator 2′ is transposed into the cell as a transmembrane nanopattern. In the embodiment in FIG. 7a, light source 6′ and detection unit 7′ are physically separate components, while in the embodiment shown in FIG. 7b, they are integral part of evanescent illuminator 2′.

FIGS. 8a and 8b show an alternative embodiment of a biomolecular detection device 1″ according to the invention. The biomolecular detection device 1 comprises an evanescent illuminator which in these particular embodiments is a total internal reflection system configured for providing a beam of coherent light at the predetermined wavelength from light source 6″ and at a predetermined angle onto the first surface of the evanescent illuminator by means of the optical coupling unit 4″. The optical coupling unit in these embodiments is a prism. In the embodiment shown in FIG. 8a, the evanescent illuminator comprises an index matching medium 11″ such as index matching oils, DMSO, glycerol, water mixtures, hydrogels, etc., and a carrier slide 12″ which contains the template nanopattern 5″. Alternatively, as shown in FIG. 8b, the evanescent illuminator can be devoid of index matching medium 11″ and a carrier slide 12″. In this case, the nanopattern 5″ is directly provided on a first surface of the optical coupling unit 4″, i.e. the prism.

### Example

### Methods and Materials

Cell culture medium DMEM High Glucose (4.5 g/l) with L-Glutamine (BioConcept, Switzerland), Lipofectamine® 2000, Opti-MEM® I (1×) Versene 1:5000 (1×), hank's balanced salt solution (HBSS), Zeozin™ was purchased from Life Technologies Europe (Zug, Switzerland). HEPES was from GERBU Biotechnik GmbH (Heidelberg, Germany), Fetal Bovine Serum (FBS) was purchased from Sigma-Aldrich Chemie GmbH (Buchs SG, Switzerland). G418 was from InvivoGen (San Diego, USA), Tissue Culture Flasks from VWR International GmbH (Dietikon, Switzerland), Biofil® Tissue culture plate 24 wells were from Axon Lab AG (Baden-Dättwil, Switzerland). Corning Costar sterile black 96 well plates, clear bottom, TC treated, Poly-D-Lysine coated were from Vitaris AG (Baar, Switzerland), Custom coated CulturPlate-96, White Opaque 96-well Microplate, Sterile and Tissue Culture Treated and ViewPlate-96, White 96-well Microplate with Clear Bottom, Sterile and Tissue Culture Treated were from Perkin Elmer (Schwerzenbach, Switzerland). TPP 6-well tissue culture plates were from Faust Laborbedarf AG (Schaffhausen, Switzerland) Coelenterazine 400a, Deep Blue C (DBC) was purchased from Cayman Chemical (Ann Arbor, Mich., United States). The GRGDSPGSC-(DBCO) peptide was custom synthesized by LifeTein, LLC (Somerset, N.J., USA). BG-GLA-NHS was obtained from BioConcept Ltd. (Alschwil, Switzerland). Azido-PEG4-NHS was obtained from Jena Bioscience (Jena, Germany). The PAA-g-PEG-NH-PhSNPPOC copolymer, used as a biocompatible coating, was provided by SuSoS. Isoproterenol hydrochloride and formoterol hemifumarate was purchased from Tocris Bioscience (Bristol, UK). ICI 118,551 hydrochloride, Fluorescein-O′-acetic acid and all other chemicals were purchased from Sigma-Aldrich Chemie GmbH (Buchs SG, Switzerland). Thin-film optical waveguides with a 145 nm Ta₂O₅ layer were obtained from Zeptosens with the in and out coupling gratings covered with a 1 μm thick layer of SiO₂ by IMT Masken and Teilungen AG (Greifensee, Switzerland).

### Expression Constructs

Beta-Arrestin 2-mPlum: mPlum coding sequence was amplified by PCR (Phusion polymerase, Finnzymes) and transferred to a pcDNA3 expression vector containing the beta-arrestin2 coding sequence.

Beta-Arrestin 2-GFP: Beta-arrestin 2 coding sequence was amplified by PCR (Phusion polymerase, Finnzymes) and transferred to a pEGFP (Clonetech) expression vector.

### Cell Lines and Cell Culture

A HEK293 cell line stably expressing the SNAP-beta2-adrenergic receptor (referred to as SNAP-β2AR) was purchased from Cisbio (Codolet, France). HEK293 stably overexpressing All HEK293 cells were cultured in DMEM supplemented with 10%-v/v fetal bovine serum and 600 μg/ml G418 with 5% CO2 at 37° C.

### Preparation of Sensor Chips

Thin-film optical waveguides were treated with a similar protocol as reported previously (Nat. Nanotechnology 2017, DOI: 10.1038/NNANO.2017.168). In short, waveguides were washed with 0.1% aqueous Tween 20, followed by ultrasound assisted washing in MilliQ water, Isopropanol and Toluene. The chips were then soaked in warm Hellmanex III for 1 min, thoroughly rinsed with MilliQ water and cleaned with highly oxidizing Piranha solution (H₂SO₄/H₂O₂ 7:3) for 30 min. After excessive washing with MilliQ water, the chips were centrifuge dried at 800 rcf for 2 min and activated by oxygen plasma. After plasma treatment, the chips were immediately immersed in the PAA-g-PEG-NH-PhSNPPOC graft copolymer coating solution (0.1 mg/ml in 1 mM HEPES pH 7.4) for 60 min. To fully passivate the layer, the chips were washed with MilliQ water and ethanol and immersed in a 25 mM solution of methyl chloroformate in anhydrous acetonitrile containing 2 equiv. of N,N-diisopropylethylamine for 5 min. The coated chips were washed with ethanol and MilliQ water, and blow dried by a nitrogen jet. Prepared sensor chips were stored in the dark at 4° C. until further use.

### Preparation of Template Nanopatterns

Template nanopatterns were prepared according to the standard reactive immersion lithography (RIL) process described (Nat. Nanotechnology 2017, DOI: 10.1038/NNANO.2017.168). Briefly, a copolymer-coated sensor chip was placed in a custom holder. The phasemask used to generate the nanopattern was aligned using an alignment help and the gap between the chip and phase mask was filled with a solution of 0.1%-v/v Hydroxyl amine in DMSO. The photolithographic exposure was conducted at 405 nm with a dose of 2000 mJ/cm² in a custom-built setup. After illumination the chip was washed with isopropanol and MilliQ water and the activated ridges were functionalized with 1 mM amine reactive SNAP-tag substrate (BG-GLA-NHS), which is covalently bound by the SNAP-tag protein. In order to increase cell adhesion to the chip, remaining PhSNPPOC groups were removed by flood exposure. The free binding sites were then functionalized with the hetero biofunctional crosslinker azido-PEG4-NHS. Finally, the chip was incubated with an azide reactive aqueous solution of 0.5 mM GRGDSPGSC-(DBCO) overnight, washed with isopropanol and MilliQ water and dried with a jet of nitrogen.

### Cell Measurements

SNAP-β₂AR cells were grown to 60-80% confluency in T25 culture flasks, washed twice with warm phosphate-buffered saline (PBS), incubated with 1× Versene for 5 min and resuspended in cell culture medium. In order to decrease baseline signal contributions from non-functional cellular debris, the cells were centrifuged at 50 rpm for 1 min and resuspended in culture media two times sequentially. The cells were seeded to reach confluency on the waveguide in an incubation chamber containing 500 μl cell culture media. Cells were only seeded when viability exceeded 90%, as determined by a Countess automated cell counter (Invitrogen). Except for the real-time establishment of the transmembrane nanopattern, seeded cells were kept in a CO₂ incubator at 37° C. for 2 h to allow cells adherence to the sensor chip (and covalent interaction of the SNAP-tag on the β₂AR with the SNAP-tag substrate on the chip). The incubation chamber containing the cells was then washed twice with warm HBSS buffer (supplemented with 20 mM HEPES, pH 7.4) adjusted for DMSO and transferred to a modified F3000 ZeptoReader (Zeptosens) which was kept at 35° C. The biomolecular detection device was then allowed to temperature equilibrate inside the ZeptoReader for 5-10 min before performing the assay. For all assays, the signal was monitored for 7 min (baseline measurement) before careful substitution of the buffer with buffer containing the β₂AR ligand and monitored for another 21 min thereafter. For the real-time establishment of the transmembrane nanopattern experiment, the measurement was performed in cell culture media supplemented with 20 mM HEPES, pH 7.4. Once the nanopattern was established, the culture media was carefully exchanged with HBSS buffer (supplemented with 20 mM HEPES, pH 7.4).

Typical instrument parameters for signal acquisition were as follows: one image every 10 s using the 635 nm laser with an integration time of 0.25-1 s depending on the intensity of the initial signal and a grey filter value of 0.001 in the illumination path of the ZeptoReader.

### Data Analysis

For all assays, the square root of the raw signal was taken. The baseline was then fitted linearly and used to de-trend the signal. Data was then displayed as a fractional change compared to baseline.

For the BRET arrestin recruitment assay the concentration-response curves with area under the curve (AUC) vs. ligand concentrations were fitted using the nonlinear regression “log(inhibitor) vs. response (three parameters)” in Graph Pad Prism to calculate the pIC50 values.

1. A biomolecular detection device for analyzing a cell, vesicle, or a cellular or vesicular component, comprising an evanescent illuminator with an optical coupling unit configured for generating an evanescent field from coherent light (L) with a predefined wavelength on a first surface of the evanescent illuminator, the first surface of the evanescent illuminator comprising a template nanopattern, containing a coherent arrangement of a plurality of predetermined lines along which membrane recognition elements for a binder structure of a transmembrane protein of the cell, vesicle, or the cellular or vesicular component are arranged, wherein the membrane recognition elements are configured to bind the binder structure of the transmembrane protein for forming a transmembrane nanopattern within the cell, vesicle, or the cellular or vesicular component based on the template nanopattern of the evanescent illuminator, such that light of the evanescent field is scattered by the cell, vesicle, or the cellular or vesicular component bound to the membrane recognition elements, and wherein the predetermined lines are arranged such that light scattered by the cell, vesicle or cellular or vesicular components bound to the membrane recognition elements constructively interferes at a predefined detection site with a difference in optical path length that is an integer multiple of the predefined wavelength of the coherent light (L).

2. The biomolecular detection device according to claim 1, wherein the evanescent illuminator comprises a carrier with a planar waveguide arranged on a surface of the carrier and an optical coupler as the optical coupling unit for coupling coherent light (L) of a predefined wavelength into the waveguide such that the coherent light propagates through the planar waveguide with an evanescent field of the coherent light (L) propagating along a first surface of the planar waveguide and wherein the first surface of the planar waveguide comprising the template nanopattern.

3. The biomolecular detection device according to claim 1, wherein the evanescent illuminator is a total internal reflection system configured for providing a beam of coherent light (L) at the predetermined wavelength and at a predetermined angle onto the first surface of the evanescent illuminator by means of the optical coupling unit, optionally by a prism.

4. The biomolecular detection device according to claim 1, wherein the membrane recognition elements are antibodies being specific to at least the binder structure of the transmembrane protein, or wherein the membrane recognition elements contain an electrophile moiety for establishing a covalent bond with the binder structure of the transmembrane protein.

5. The biomolecular detection device according to claim 1, wherein the plurality of predetermined lines comprises curved lines with a curvature configured such that light of the evanescent field scattered by the cell, vesicle, or the cellular or vesicular component bound to the membrane recognition elements interferes at the predefined detection site.

6. The biomolecular detection device according to claim 1, wherein the first surface of the evanescent illuminator comprises a cell adhesive.

7. The biomolecular detection device according to claim 1, wherein at least one cell, vesicle, or cellular or vesicular component is bound via the binder structure of the transmembrane protein to the membrane recognition elements.

8. The biomolecular detection device according to claim 1, wherein the predetermined lines are separated from each other by areas devoid of membrane recognition elements and wherein the areas devoid of membrane recognition elements are configured to inverse an optical modulation, which is induced by the binding of a structural recognition element to the binder structure of the transmembrane protein, such that the signal obtained from binding of the structural recognition element to the binder structure of the transmembrane protein is provided in a different operating window of the biomolecular detection device, wherein the different operating window is at an intensity close to zero.

9. (canceled)

10. A method of detecting molecular interactions associated with cells, vesicles, or cellular or vesicular components, comprising:
providing the biomolecular detection device according to claim 1;
applying a cell or vesicle to the membrane recognition elements, wherein the cell or the vesicle comprises a membrane and at least one transmembrane protein with an extracellular or extravesicular binder structure, optionally where
at least one transmembrane protein is laterally diffused along the membrane;
aligning the al least one transmembrane protein of the cell or the vesicle according to the template nanopattern of the first surface of the evanescent illuminator, such that a transmembrane nanopattern is formed in the membrane of the cell or the vesicle, wherein the transmembrane pattern corresponds at least partially to the template nanopattern of the first surface of the evanescent illuminator;
generating a beam of coherent light at a predefined beam generation location relative to the plurality of predetermined lines, the beam of coherent light having a predefined wavelength and being incident on the membrane recognition elements with the bound transmembrane protein in a manner that diffracted portions of the incident beam of coherent light constructively interfere at the predefined detection site relative to the plurality of predetermined lines with a difference in optical path length that is an integer multiple of the predefined wavelength of the coherent light to provide a signal representative of the membrane recognition elements with the transmembrane protein of a cell, vesicle, or cellular or vesicular component bound thereto at the predefined detection site; and
measuring the signal representative for the membrane recognition elements with the transmembrane protein of a cell, vesicle, or cellular or vesicular component bound thereto.

11. The method according to claim 10, further comprising the step of comparing the measured signal representative of the membrane recognition elements with the transmembrane of a cell, vesicle, or cellular or vesicular component bound thereto, with an unbound signal representative of only the membrane recognition elements.

12. The method of claim 10, wherein a cell is applied to the membrane recognition elements and wherein before generating the beam of coherent light the cell is modified such that only parts of the cell membrane remain on the biomolecular detection device.

13. The method according to claim 10, wherein the binder structure of the transmembrane protein is specific to an antibody being arranged along the predefined lines of the evanescent illuminator of the biomolecular detection device.

14. The method according to claim 10, wherein a protein of interest of the biomolecular interaction comprises a high-mass moiety.

15. The method according to claim 10, wherein additionally, optionally simultaneously, a fluorescent and/or bioluminescent signal is recorded.

16. A method for generating a transmembrane nanopattern within a cell, vesicle, or cellular or vesicular component, the method comprising:
providing the biomolecular detection device according to claim 1;
applying a cell or a vesicle is applied to the membrane recognition elements, wherein the cell or the vesicle comprises a membrane and at least one transmembrane protein with an extracellular or extravascular binder structure;
optionally laterally diffusing at least one transmembrane protein along the membrane; and
binding the extracellular binding structure of the at least one transmembrane protein to any of the membrane recognition elements and aligning the at least one transmembrane protein according to the template nanopattern of the first surface of the evanescent illuminator, such that a transmembrane nanopattern is formed in the membrane of the cell or the vesicle, wherein the transmembrane pattern corresponds at least partially to the template nanopattern of the first surface of the evanescent illuminator.

17. The method according to claim 16, wherein the binder structure of the transmembrane protein is specific to an antibody being arranged along the predefined lines of the evanescent illuminator of the biomolecular detection device.

18. The method according to claim 12, wherein after aligning the transmembrane protein and forming of the transmembrane nanopattern, the cell is modified such that only parts of the cell membrane remain on the biomolecular detection device.
